(19) United States
(12) Patent Application Publication
(10) Pub. No.: US 2017/0150899 A1
(43) Pub. Date: Jun. 1, 2017

(54) ELECTRONIC DEVICE FOR ANALYZING BIO-ELECTRICAL IMPEDANCE USING CALIBRATED CURRENT

(71) Applicant: SAMSUNG ELECTRONICS CO., LTD., SUWON-SI (KR)

(72) Inventor: BYUNGKI MOON, SEOUL (KR)

(21) Appl. No.: 15/345,577

(22) Filed: Nov. 8, 2016

(30) Foreign Application Priority Data
Nov. 26, 2015 (KR) 10-2015-0166322

(51) Int. Cl. A61B 5/053; A61B 5/00

(52) U.S. Cl. CPC A61B 5/0537; A61B 5/4869; A61B 5/4872; A61B 5/0022

(57) ABSTRACT

An electronic device for analyzing bio-electrical impedance includes a current generator, a calibration load, a switch circuit, and a processor. The current generator generates a source current. The calibration load includes an impedance component. The switch circuit provides the source current to the calibration load or outputs the source current to an outside of the electronic device. The processor controls the switch circuit such that the source current is provided to the calibration load in response to a request for analyzing the bio-electrical impedance, and controls the switch circuit such that the source current is output to the outside of the electronic device when a voltage value of a test voltage that is provided between both ends of the calibration load according to the source current is included in a reference range.

### CROSS-REFERENCE TO RELATED APPLICATION

A claim of priority under 35 U.S.C. §119 is made to Korean Patent Application No. 10-2015-0166322, filed on Nov. 26, 2015, in Korean Intellectual Property Office, the entire contents of which are hereby incorporated by reference.

### BACKGROUND

The inventive concept herein relates to an electronic device, and more particularly, to an electronic device configured to process an electrical signal to analyze bioelectrical impedance.

A bio-electrical impedance analysis device is an example of an electronic device that may for example be used to analyze impedance of a human body. The impedance of the human body may be related to body composition, such as body fat, muscle, and so on. Body composition may thus be understood using a bio-electrical impedance analysis device. For example, information associated with body composition may be referred to so as to understand health conditions of a person or to perform medical treatment.

Some bio-electrical impedance analysis devices inject current into the human body, and obtain information associated with impedance of the human body based on the injected current. However, too strong a current may pose serious threat. Thus, for safety, bio-electrical impedance analysis devices need to be accurately controlled. Also, the current output from bio-electrical impedance analysis devices should have proper intensity to enable accurate analysis of body composition.

### SUMMARY

The present inventive concept relates to an electronic device that is configured to analyze bio-electrical impedance. The electronic device may analyze the bio-electrical impedance using a “calibrated” current. The intensity of the current may be calibrated to a safe value and/or a desired value.

Embodiments of the inventive concept provide an electronic device configured to analyze bio-electrical impedance. The electronic device includes a current generator, a calibration load, a switch circuit, and a processor. The current generator is configured to generate a source current. The calibration load includes an impedance component. The switch circuit is configured to selectively provide the source current to the calibration load, and to output the source current externally of the electronic device. The processor is configured to control the switch circuit to provide the source current to the calibration load in response to a request for analyzing the bio-electrical impedance, and to output the source current externally of the electronic device upon determination that a voltage value of a test voltage is within a reference range. The test voltage is provided between both ends of the calibration load responsive to the source current.

Embodiments of the inventive concept provide an electronic device configured to analyze bio-electrical impedance. The electronic device includes a calibration load, a switch circuit, a comparator, and a controller. The calibration load includes an impedance component. The switch circuit is configured to selectively provide a source current to the calibration load and to output the source current externally of the electronic device. The source current is generated by a current generator. The comparator is configured to compare a voltage value of a test voltage with one or more reference values. The test voltage is provided between both ends of the calibration load responsive to the source current provided from the switch circuit. The one or more reference values are within a reference range. The controller is configured to control an operation of the switch circuit and an intensity of the source current generated by the current generator, based on an output of the comparator.

Embodiments of the inventive concept provide an electronic device configured to analyze bio-electrical impedance. The electronic device includes a current generator configured to generate a source current; a calibration load including an impedance component and configured to provide a test voltage responsive to the source current, wherein an impedance value of the impedance component corresponds to an estimated impedance value of the bio-electrical impedance; a pair of electrodes connected to an outside of the electronic device; and a processor configured to control the current generator to adjust an intensity of the source current responsive to the test voltage, to output the source current having the adjusted intensity externally of the electronic device, and to obtain information associated with the bio-electrical impedance based on a voltage externally applied to the pair of electrodes responsive to the output source current.

### BRIEF DESCRIPTION OF THE DRAWINGS

The forgoing and other objects, features, and advantages of the present disclosure will be described below in more detail with reference to the accompanying drawings of non-limiting embodiments in which like reference characters may refer to like parts throughout the different drawings.

FIG. 1 illustrates a conceptual diagram of a bio-electrical impedance analysis system that includes an electronic device according to embodiments of the inventive concept.

FIG. 2 illustrates a table describing a relationship between a measurement value obtained by an electronic device of FIG. 1 and a body composition.

FIG. 3 illustrates a block diagram of an electronic device according to embodiments of the inventive concept.

FIG. 4 illustrates a state diagram describing an operation of an electronic device of FIG. 3.

FIG. 5 illustrates a conceptual diagram describing a test voltage obtained in a calibration mode, and intensity of a source current adjusted in the calibration mode.

FIG. 6 illustrates a flowchart describing an operation of an electronic device of FIG. 3.

FIGS. 7, 8 and 9 illustrate conceptual diagrams for describing operations of an electronic device of FIG. 3.

FIG. 10 illustrates a flowchart describing an operation of an electronic device of FIG. 3.

FIGS. 11, 12 and 13 illustrate block diagrams of electronic devices according to embodiments of the inventive concept.

FIG. 14 illustrates a block diagram of a mobile electronic device that includes a bio-electrical impedance analysis circuit/chip according to embodiments of the inventive concept.

### DETAILED DESCRIPTION

The inventive concept should not be construed as limited to the “example” embodiments set forth herein, and may be embodied in different forms. Hereinafter, example embodiments of the inventive concept will be described below with reference to the attached drawings.

As is traditional in the field of the inventive concepts, embodiments may be described and illustrated in terms of blocks which carry out a described function or functions. These blocks, which may be referred to herein as units or modules or the like, are physically implemented by analog and/or digital circuits such as logic gates, integrated circuits, microprocessors, microcontrollers, memory circuits, passive electronic components, active electronic components, optical components, hardwired circuits and the like, and may optionally be driven by firmware and/or software. The circuits may, for example, be embodied in one or more semiconductor chips, or on substrate supports such as printed circuit boards and the like. The circuits constituting a block may be implemented by dedicated hardware, or by a processor (e.g., one or more programmed microprocessors and associated circuitry), or by a combination of dedicated hardware to perform some functions of the block and a processor to perform other functions of the block. Each block of the embodiments may be physically separated into two or more interacting and discrete blocks without departing from the scope of the inventive concepts. Likewise, the blocks of the embodiments may be physically combined into more complex blocks without departing from the scope of the inventive concepts.

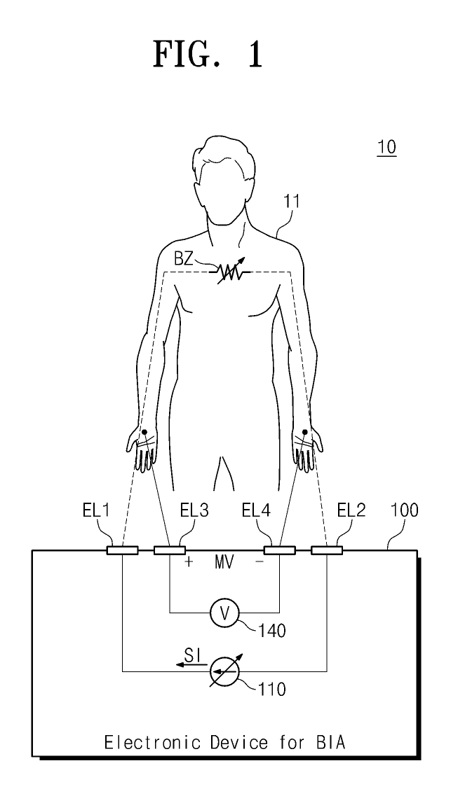

FIG. 1 illustrates a conceptual diagram of a bio-electrical impedance analysis system that includes an electronic device according to embodiments of the inventive concept. For example, bio-electrical impedance analysis (BIA) system 10 includes body 11 and electronic device 100.

Body 11 may be a human body, but in other embodiments may be the body of other creatures such as animals or the like. Body 11 may include bio-electrical impedance BZ. The bio-electrical impedance BZ may be related to body composition, such as body fat, muscle, and so on. Electrical current may easily flow or may not flow well through the body 11, depending on the body composition. The bio-electrical impedance BZ may have an impedance value that is variable depending on the body composition.

The electronic device 100 may be used to analyze the bio-electrical impedance BZ. In some embodiments, the electronic device 100 may be configured to directly measure the bio-electrical impedance BZ. In other embodiments, the electronic device 100 may indirectly obtain information of the bio-electrical impedance BZ.

The electronic device 100 includes a current source 110. The current source 110 outputs source current SI. The current source 110 may generate the source current SI using power supplied from a power supply circuit/device (not illustrated in FIG. 1) that is provided inside the electronic device 100 or provided separately from the electronic device 100.

The source current SI is output from the electronic device 100, and provided to the body 11. The electronic device 100 includes electrodes EL1 and EL2 to be connected with the body 11. The electrodes EL1 and EL2 may be connected to (e.g., attached on) a part (e.g., palms, a wrist, a chest, and so on) of the body 11. The source current SI is injected into the body 11 through the electrode EL1. The source current SI flows through the bio-electrical impedance BZ, is output from the body 11, and is provided to the electronic device 100 through the electrode EL2.

It should be understood by those skilled in the art that a voltage is provided between (or across) both ends of a resistor (or impedance) when current flows through the resistor (or the impedance). Thus, when the source current SI flows through the bio-electrical impedance BZ, a measurement voltage MV exists between parts of the body 11 to which the electrodes EL1 and EL2 are connected. The electronic device 100 includes a voltage meter circuit 140 to measure the measurement voltage MV.

The electronic device 100 includes electrodes EL3 and EL4. The electrode EL3 is connected to the part of the body 11 to which the electrode EL1 is connected, and the electrode EL4 is connected to the part of the body 11 to which the electrode EL2 is connected. The voltage meter circuit 140 is connected between the electrodes EL3 and EL4. Thus, the voltage measurement circuit 140 may measure the measurement voltage MV applied between the electrodes EL3 and EL4.

It should be understood by those skilled in the art that voltage amplitude is proportional to the product of current intensity and a value of a resistor (or impedance). Thus, when amplitude of a measurement voltage MV applied according to the source current SI is measured, an impedance value of the bio-electrical impedance BZ may be calculated based on intensity of the source current SI and the amplitude of the measurement voltage MV. For example, the electronic device 100 may further include an operation processing circuit/device (not illustrated in FIG. 1) to calculate the impedance value of the bio-electrical impedance BZ based on the intensity of the source current SI and the amplitude of the measurement voltage MV.

The electronic device 100 may obtain information (e.g., an impedance value) of the bio-electrical impedance BZ. Further, the electronic device 100 may analyze the bio-electrical impedance BZ to obtain information associated with the body composition of the body 11. Thus, the electronic device 100 may be used to understand the body composition of the body 11. For example, the information associated with the body composition may be referred to so as to understand health conditions of the body 11 or perform medical treatment.

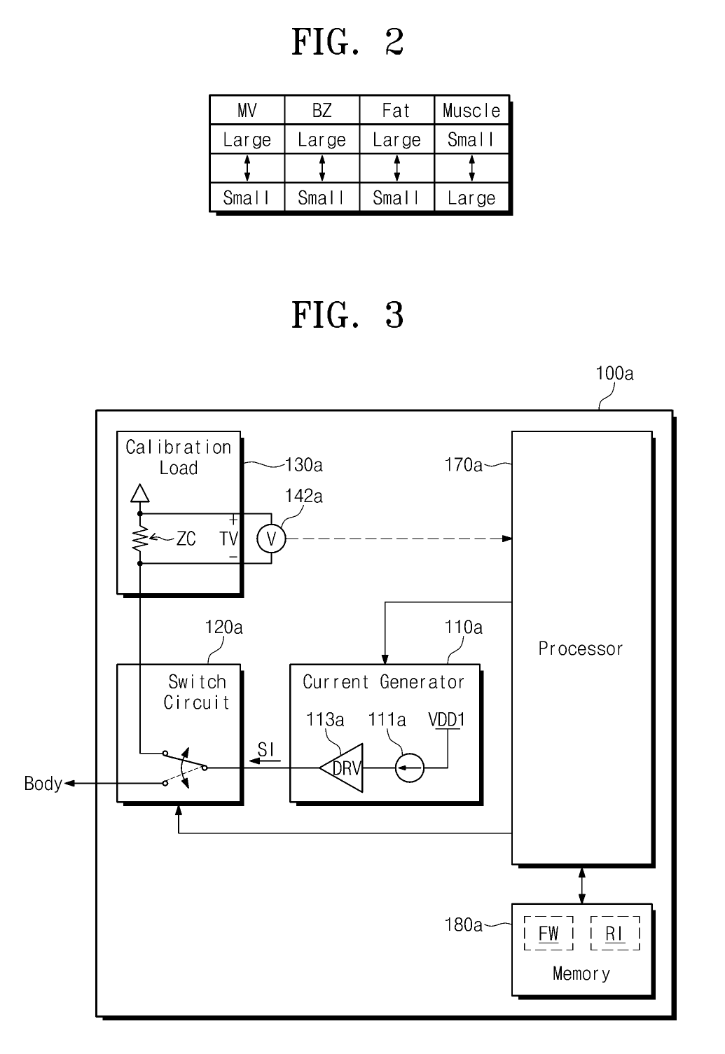

FIG. 2 illustrates a table describing a relationship between a measurement value obtained by an electronic device of FIG. 1 and body composition. To help better understanding, FIG. 1 will be referred to together with FIG. 2.

As described with reference to FIG. 1, the electronic device 100 measures a measurement voltage MV and calculates an impedance value of bio-electrical impedance BZ based on amplitude of the measurement voltage MV.

For example, when intensity of the source current SI generated by the current source 110 is constant, the impedance value of the bio-electrical impedance BZ may be proportional to the amplitude of the measurement voltage MV. This is because the amplitude of the measurement voltage MV is proportional to the intensity of the source current SI and the impedance value of the bio-electrical impedance BZ. Thus, as the amplitude of the measurement voltage MV becomes larger, the impedance value of the bio-electrical impedance BZ becomes larger. On the other hand, as the amplitude of the measurement voltage MV becomes smaller, the impedance value of the bio-electrical impedance BZ becomes smaller.

Meanwhile, as described with reference to FIG. 1, the body 11 may include various body compositions. For example, the body 11 may include body fat and muscle. The body 11 may further include other ingredients.

For example, the body fat may be a non-conductive ingredient that interrupts current flow. Thus, when the impedance value of the bio-electrical impedance BZ is large, the body 11 may include a large amount of body fat. On the other hand, when the impedance value of the bio-electrical impedance BZ is small, the body 11 may include a small amount of body fat. This is because current flows better through bio-electrical impedance BZ having smaller impedance value.

Muscle may be a conductive ingredient through which current flows relatively easily. Thus, when the impedance value of the bio-electrical impedance BZ is large, the body 11 may include a small amount of muscle. On the other hand, when the impedance value of the bio-electrical impedance BZ is small, the body 11 may include a large amount of muscle.

In such a manner, the impedance value of the bio-electrical impedance BZ may be measured to understand the body composition of the body 11. This is because the bio-electrical impedance BZ may have an impedance value that varies depending on the body composition. The electronic device 100 may obtain information of the bio-electrical impedance BZ based on the intensity of the source current SI and the amplitude of the measurement voltage MV. Further, the electronic device 100 may be used to understand the body composition based on the obtained information.

The electronic device 100 injects the source current SI into the body 11 to obtain the information of the bio-electrical impedance BZ. However, source current SI having too strong intensity may damage the body 11. Thus, in embodiments of the inventive concept, the electronic device 100 is accurately controlled for safety of the body 11, and/or the source current SI has proper intensity (for instance, intensity that is not immoderately weak) to accurately analyze the body composition.

The electronic device 100 in embodiments of the inventive concept outputs a “calibrated” source current SI. The source current SI thus has intensity calibrated to a safe value and/or a desired value. Damage to the body 11 due to excessively strong source current SI may be prevented. Further, the calibrated source current SI has proper intensity to accurately analyze the body composition.

FIG. 3 illustrates a block diagram of an electronic device according to embodiments of the inventive concept. For example, the electronic device 100 of FIG. 1 may include an electronic device 100a of FIG. 3. The electronic device 100a may be used to analyze the bio-electrical impedance BZ of FIG. 1.

Referring to FIGS. 1 and 3, the electronic device 100a includes a current generator 110a, a switch circuit 120a, a calibration load 130a, a voltage meter circuit 142a, a processor 170a, and a memory 180a. The configuration of the electronic device 100a illustrated in FIG. 3 should not be construed as limiting, and in other embodiments the electronic device 100a may not include at least one of components illustrated in FIG. 3 and/or may further include other components that are not illustrated in FIG. 3.

The current generator 110a generates a source current SI which is output to obtain information of the bio-electrical impedance BZ. The source current SI is injected into the body 11.

The current generator 110a includes a current source 111a and a current driver 113a. The current source 111a may generate current using power source voltage VDD1. The power source voltage VDD1 may be supplied from a power supply circuit/device (not illustrated in FIG. 3) that is provided inside the electronic device 100a or provided separately from the electronic device 100a.

The current driver 113a drives an output of the current provided from the current source 111a. Accordingly, the current driver 113a outputs the source current SI. For example, the current driver 113a may amplify the current provided from the current source 111a. An amplification level of the current driver 113a may be variable. Thus, intensity of the source current SI may be adjusted. The current driver 113a may for example include a programmable/adjustable gain amplifier.

The switch circuit 120a receives the source current SI from the current generator 110a. The switch circuit 120a may connect the current generator 110a to the calibration load 130a, to provide the source current SI to the calibration load 130a. Alternatively, the switch circuit 120a may connect the current generator 110a to the outside of the electronic device 100a (e.g., the body 11), to output the source current SI to the outside of the electronic device 100a. In other words, the switch circuit 120a may output the source current SI externally of the electronic device 100a to the body. The switch circuit 120a may thus selectively transmit the source current SI to one of the outside of the electronic device 100a (e.g., the body 11) and the calibration load 130a.

The calibration load 130a includes an impedance component ZC. The impedance component ZC has an impedance value, and may enable current to flow there through easily or with difficulty, depending on the impedance value. In some embodiments, the impedance component ZC may have an identical or similar impedance value to the bio-electrical impedance BZ of the body 11. For example, the impedance value of the impedance component ZC may correspond to an estimated impedance value of the bio-electrical impedance BZ. This may mean that the impedance component ZC may be implemented to have an electrical characteristic that is identical or similar to that of the body 11.

When the source current SI flows through the impedance component ZC of the calibration load 130a, a test voltage TV is provided between both ends of the calibration load 130a. That is, the test voltage TV is provided between the both opposite ends of the calibration load 130a according to (or responsive to) the source current SI. The voltage meter circuit 142a is connected between both ends of the impedance component ZC of the calibration load 130a. Accordingly, the voltage meter circuit 142a may be used to measure amplitude of the test voltage TV. The voltage meter circuit 142a may be implemented in one circuit together with the voltage meter circuit 140, or may be provided separately from the voltage meter circuit 140.

As will be described later, the electronic device 100a may operate in one of a “calibration mode” or a “measurement mode”. The calibration mode may be provided to calibrate the intensity of the source current SI.

In the calibration mode, the source current SI is provided to the calibration load 130a through the switch circuit 120a. While the source current SI is provided to the impedance component ZC of the calibration load 130a, it may be checked whether the intensity of the source current SI is proper or not. Herein, the proper intensity of the source current SI may mean safe intensity that does not damage the body 11. Additionally or alternatively, the proper intensity of the source current SI may mean intensity determined to accurately measure the impedance value of the bio-electrical impedance BZ and to accurately analyze the body composition of the body 11. Whether the intensity of the source current SI is proper may be determined based on the amplitude of the test voltage TV. When the intensity of the source current SI is not proper, the intensity of the source current SI may be calibrated as will be described later.

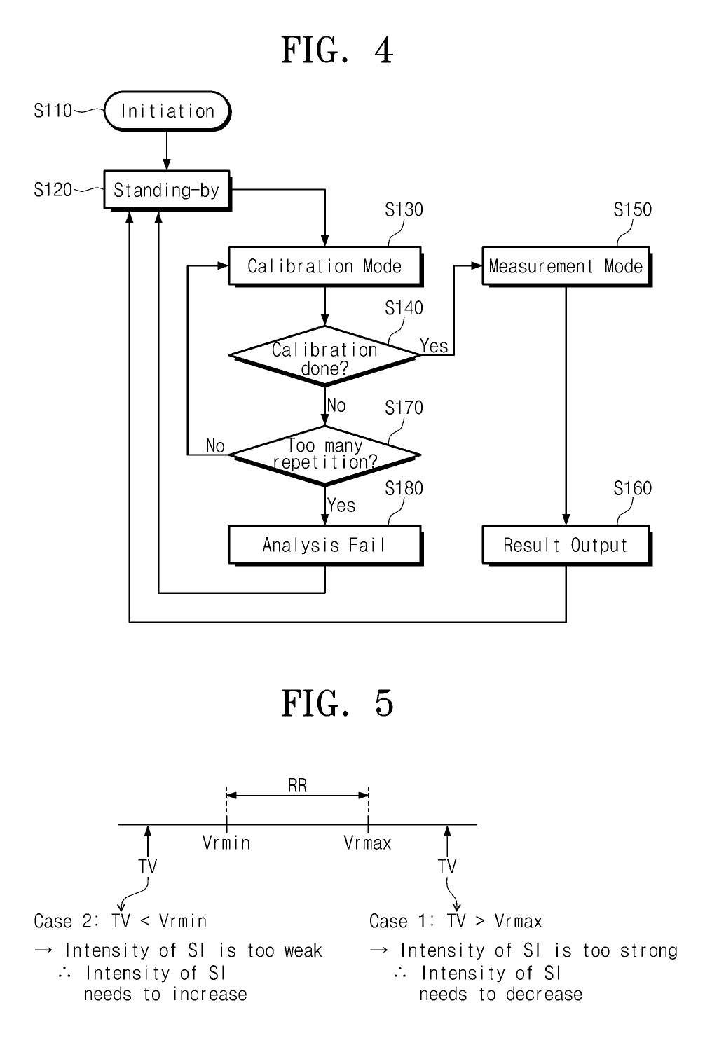

On the other hand, when the intensity of the source current SI is proper, the electronic device 100a may operate in the measurement mode. In the measurement mode, the source current SI is output to the outside of the electronic device 100a (e.g., the body 11) through the switch circuit 120a. In the measurement mode, the electronic device 100a, as described with reference to FIG. 1, may obtain information of the bio-electrical impedance BZ based on the output source current SI. The operation modes of the electronic device 100a will be described in further detail with reference to FIGS. 4 through 10.

As described above, the impedance component ZC has an impedance value that is identical or similar to the bio-electrical impedance BZ. In some embodiments, the impedance component ZC may include a variable impedance component. For example, the electronic device 100a may store information of a specific height of the body 11 and a standard weight corresponding to the specific height, for instance, in the memory 180a, in advance before operating the electronic device 100a. Further, the electronic device 100a may store information of a standard impedance value corresponding to the specific height and the standard weight, for instance, in the memory 180a, in advance before operating the electronic device 100a. For example, the electronic device 100a may receive information of height and weight from a user, and may adjust the impedance value of the impedance component ZC based on the received information. In such example embodiments, the impedance component ZC may have an optimal electrical characteristic that is identical or similar to that of the body 11.

In other embodiments, the impedance component ZC may have a fixed impedance value. In still further embodiments, the impedance component ZC may have an impedance value that is adjustable depending on a heart beat rate or body temperature of a user. These embodiments may be variously changed or modified.

The processor 170a manages the overall operations of the electronic device 100a. For example, the processor 170a may process various arithmetic operations and/or various logic operations required to operate the electronic device 100a. The processor 170a may include one or more processor cores that are capable of processing various operations. The processor 170a may include a special-purposed logic circuit, such as field programmable gate array (FPGA), application specific integrated circuits (ASICs), and/or the like.

For example, the processor 170a may be configured to execute an instruction code. The processor 170a may interpret and understand an instruction code of software and/or firmware, and perform an operation based on the instruction code, and output an operation result. The processor 170a may manage an operation of the electronic device 100a based on the operation result. The operations of the processor 170a that will be described below may be performed based on one or more instruction codes of software and/or firmware.

The processor 170a controls operations of the current generator 110a. For example, the processor 170a may control the current generator 110a such that the intensity of the source current SI is adjusted. For example, when the amplification level of the current driver 113a is variable, the processor 170a may control the amplification level of the current driver 113a to adjust the intensity of the source current SI.

The processor 170a controls operations of the switch circuit 120a. For example, the processor 170a may receive an amplitude value of test voltage TV from the voltage meter circuit 142a. The processor 170a may determine whether the intensity of the source current SI is proper, based on the amplitude of the test voltage TV.

When it is determined that the intensity of the source current SI is not proper, the processor 170a may operate the electronic device 170a in the calibration mode. In the calibration mode, the processor 170a controls the switch circuit 120a such that the source current SI is provided to the calibration load 130a. On the other hand, when it is determined that the intensity of the source current SI is proper, the processor 170a may operate the electronic device 170a in the measurement mode. In the measurement mode, the processor 170a controls the switch circuit 120a such that the source current SI is output to the outside of the electronic device 100a. The processor 170a may provide a control signal(s) to the current generator 110a and the switch circuit 120a to control the current generator 110a and the switch circuit 120a.

The memory 180a may store various data that is used to operate the electronic device 100a. For example, the memory 180a may include a volatile memory (e.g., static random access memory (SRAM), dynamic RAM (DRAM), synchronous DRAM (SDRAM), and/or the like) and/or a nonvolatile memory (e.g., phase-change RAM (PRAM), magnetic RAM (MRAM), resistive RAM (ReRAM), ferroelectric RAM (FRAM), and/or the like). The memory 180a may include homogeneous or heterogeneous memories.

The memory 180a may store data processed or to be processed by the processor 170a. For example, the memory 180a may store one or more instruction codes of firmware FW that define operations of the processor 170a. The processor 170a may receive the instruction codes of firmware from the memory 180a. The processor 170a may control the operation of the electronic device 100a based on the instruction codes.

The memory 180a may store reference information RI. The reference information RI may include information that is referred to in the calibration mode. For example, in the calibration mode, the processor 170a may determine whether the amplitude of the test voltage TV is proper, with reference to the reference information RI. The processor 170a may determine whether the intensity of the source current SI is proper, according to the amplitude of the test voltage TV. This will be described in more detail with reference to FIG. 5.

FIG. 4 illustrates a state diagram describing an operation of an electronic device of FIG. 3. To help better understanding, FIGS. 1 and 3 will be referred to together with FIG. 4.

In operation S110, an operation of the electronic device 100a is initiated. For example, a user of the electronic device 100a may turn the power of the electronic device 100a on. As the power is supplied to the electronic device 100a, the electronic device 100a may begin to operate. Alternatively, as an operation of the electronic device 100a is reset, an operation of the electronic device 100a may be initiated. For example, when an error occurs in an operation of the electronic device 100a, a user or the processor 170a may reset the electronic device 100a.

In operation S120, the electronic device 100a enters a state of stand-by. The electronic device 100a may be in a state of stand-by before an operation for analyzing the bio-electrical impedance BZ is performed. The electronic device 100a may be in a state of stand-by until a request for analyzing the bio-electrical impedance BZ is provided. Herein, “standing-by” may mean that the electronic device 100a does not perform any operation. Alternatively, “standing-by” may mean that the electronic device 100a performs operations other than analyzing the bio-electrical impedance BZ.

The electronic device 100a may receive the request for analyzing the bio-electrical impedance BZ. A user of the electronic device 100a may input the request for analyzing to the electronic device 100a through a user interface of the electronic device 100a. Alternatively, when a specific condition is satisfied, the request for analyzing may occur inside the electronic device 100a.

In operation S130, the electronic device 100a begins to operate in the calibration mode, in response to the request for analyzing the bio-electrical impedance BZ. That is, the processor 170a operates the electronic device 100a in the calibration mode. In embodiments of the inventive concept, the electronic device 100a may operate in the calibration mode first, instead of immediately obtaining information of the bio-electrical impedance BZ in response to the request of analyzing the bio-electrical impedance BZ.

As described with reference to FIG. 3, the source current SI having strong intensity may damage the body 11. Moreover, when the source current SI having immoderately weak intensity is output from the electronic device 100a, the measurement voltage MV may not be well measured and the information of the bio-electrical impedance BZ may not be accurately obtained.

The intensity of the source current SI may vary due to various causes. For example, an error that occurs in a manufacturing process of the electronic device 100a may cause a malfunction of the current generator 110a, and thus the intensity of the source current SI may not be accurately controlled. For example, the source current SI may have improper intensity depending on an environment (e.g., temperature, humidity, device life, and so on) where the electronic device 100a operates. In embodiments, the calibration mode may be provided to calibrate the intensity of the source current SI to a safe value and/or a desired value. The calibration mode will be described in further detail with reference to FIGS. 5 through 10.

In operation S140, the processor 170a of the electronic device 100a determines whether a calibration of the intensity of the source current SI is completed. When the calibration is completed, an operation of the electronic device 100a transits to operation S150. In operation S150, the processor 170a operates the electronic device 100a in the measurement mode.

After operation S150, in operation S160, the electronic device 100a operating in the measurement mode measures the voltage value of the measurement voltage MV that exists between the electrodes EL3 and EL4 according to the source current SI using voltage meter circuit 140. Further, processor 170a of the electronic device 100a obtains the information (e.g., an impedance value) of the bio-electrical impedance BZ based on the measured voltage value. The electronic device 100a may output various results, such as the information of the bio-electrical impedance BZ and/or additional information (e.g., an amount of body fat, an amount of muscle, etc.) obtained by processor 170a analyzing the information of the bio-electrical impedance BZ. After the results are output in operation S160, the electronic device 100a enters the state of stand-by in operation S120.

In some embodiments, when it is determined that the calibration is not completed in operation S140, an operation of the electronic device 100a transits to operation S170. In operation S170, processor 170a of the electronic device 100a determines whether the calibration operation in operation S130 for calibrating the intensity of the source current SI has been repeated too many times (e.g., whether the number of repetitions of the calibration operation is larger than a reference number).

For example, when there is a severe error in the electronic device 100a, it may be difficult to effectively calibrate the intensity of the source current SI. When the calibration operation is repeated even though it is difficult to calibrate the source current SI, a “deadlock” may occur in an operation of the electronic device 100a. To avoid the deadlock, operation S170 may be provided. The reference number may have any appropriate value to avoid the deadlock. For example, a value of the reference number may be stored in the memory 180a, and may be referred by the processor 170a. Alternatively or additionally, the reference number may be inserted into the instruction code of firmware FW, and may be processed by the processor 170a.

When it is determined that the calibration operation has been repeated an amount of times less than the reference number in operation S170, an operation of the electronic device 100a transits to operation S130. In operation S130, the electronic device 100a operates in the calibration mode. The operations S130, S140 and S170 may be repeated until the calibration operation is completed. Alternatively, the operations S130, S140 and S170 may be repeated until the calibration operation is repeated as many times as the reference number.

When the calibration operation is repeated as many times as the reference number (in other words, when it is estimated that the calibration operation is repeated too much and deadlock has occurred in an operation of the electronic device 100a), an operation of the electronic device 100a transits to operation S180. In operation S180, the processor 170a of the electronic device 100a determines that analyzing the bio-electrical impedance BZ has failed. For example, the electronic device 100a may provide a user with any corresponding result indicating an analysis failure. After the result of analysis failure is output in operation S180, the electronic device 100a enters the state of stand-by in operation S120.

FIG. 5 illustrates a conceptual diagram describing a test voltage obtained in a calibration mode of FIG. 4 and intensity of a source current adjusted in the calibration mode of FIG. 4. To help better understanding, FIGS. 1 and 3 will be referred to together with FIG. 5.

Reference information RI stored in the memory 180a may include information associated with a reference range RR. The reference range RR may be a reference interval including an upper limit Vrmax and a lower limit Vrmin. The reference range RR may be designed to include a voltage value of the test voltage TV that is provided between both ends of the calibration load 130a when the source current SI having “proper intensity” flows through the calibration load 130a. The processor 170a of the electronic device 100a compares the reference range RR with the voltage value of the test voltage TV to determine whether the intensity of the source current SI is proper.

In the following description, it will be assumed that the proper intensity of the source current SI is 1 microampere (μA). This may mean that the body 11 may not be damaged and the bio-electrical impedance BZ may be accurately analyzed when the intensity of the source current SI is about 1 μA. This assumption is merely provided to help better understanding, and should not be construed as limiting.

For example, when the calibration load 130a has an impedance value of 1 mega-ohm (MΩ) and the source current SI of 1 μA flows through the calibration load 130a, the amplitude of the test voltage TV is 1 volt (V). In this example, the reference range RR may be designed to include a voltage value of 1V.

Further, for example, the upper limit Vrmax and the lower limit Vrmin of the reference range RR may be selected to allow a margin of 10 percent (%) with respect to a voltage value of 1V (e.g., the upper limit Vrmax of the reference range RR may be selected to have a voltage value of 1.1V (=1V+1V×10%), and the lower limit Vrmin of the reference range RR may be selected to have a voltage value of 0.9V (=1V−1V×10%)). That is, the upper limit Vrmax and the lower limit Vrmin of the reference range RR may be properly selected to include the voltage value of 1V. The upper limit Vrmax and the lower limit Vrmin may be provided to cover a small error that may occur during numerical measurement.

However, the above examples should not be construed as limiting. In other embodiments, current intensity, an impedance value, a voltage value, and/or selected values of the upper limit Vrmax and the lower limit Vrmin may be variously changed or modified. In the embodiments, the reference range RR may be designed to include the amplitude of the test voltage TV that corresponds to the source current SI having the proper intensity. Further, the upper limit Vrmax and the lower limit Vrmin may be selected such that the reference range RR includes the amplitude of the test voltage TV that corresponds to the source current SI having proper intensity.

Accordingly, the reference range RR may mean amplitude of the test voltage TV that is expected to be measured in response to a specific impedance value of the calibration load 130a. For example, when the calibration load 130a has an impedance value of 1 MΩ, the electronic device 100a may expect, with reference to the reference range RR, that the test voltage TV having amplitude of about 1V (e.g., amplitude between 0.9V and 1.1V) will be measured. In some embodiments, the reference information RI may include information of plural reference ranges RR that corresponds respectively to plural impedance values of the calibration load 130a.

The information of the reference range RR may be prepared by a designer in advance before operating the electronic device 100a (e.g., when the electronic device 100a is manufactured). Alternatively or additionally, the information of the reference range RR may be prepared by a designer or a user after the electronic device 100a is manufactured. In some cases, the electronic device 100a may learn any proper reference range RR while it is operating, and the information of the reference range RR may be updated according to learning of the electronic device 100a.

As described with reference to FIG. 3, the electronic device 100a may measure the voltage value of the test voltage TV using the voltage meter circuit 142a. The electronic device 100a (more specifically, the processor 170a) may compare the voltage value of the test voltage TV with the reference range RR. Based on the comparison result, the electronic device 100a may determine whether the intensity of the source current SI is proper.

For example, when the test voltage TV is measured to have a voltage value of 1V, the electronic device 100a may understand that the source current SI has “proper intensity” of 1 μA. For example, when the test voltage TV is measured to have a voltage value between 0.9V and 1.1V, the electronic device 100a may understand that the source current SI has intensity of about 1 μA with a small error.

On the other hand, in some cases, the test voltage TV may have a voltage value that exceeds 1.1V or does not reach 0.9V. In this case, the electronic device 100a may understand that the source current SI has intensity having a great difference from 1 μA. For example, when the test voltage TV has a voltage value that exceeds 1.1V, the electronic device 100a may understand that the source current SI has intensity stronger than 1 μA. On the other hand, when the test voltage TV has a voltage value that does not reach 0.9V, the electronic device 100a may understand that the source current SI has intensity weaker than 1 μA. In the embodiments, the processor 170a of the electronic device 100a may determine whether the source current SI has proper intensity, based on the voltage value of the test voltage TV.

A first case will be described where the voltage value of the test voltage TV is greater than the upper limit Vrmax of the reference range RR. For example, when the test voltage TV has a voltage value that exceeds 1.1V, the source current SI may have intensity stronger than 1 μA. (This is because the intensity of the source current SI is proportional to the voltage value of the test voltage TV). As described above, the source current SI having strong intensity may damage the body 11. Thus, for the first case, the intensity of the source current SI may be adjusted to decrease, and the electronic device 100a may operate in the calibration mode to decrease the intensity of the source current SI.

A second case will be described where the voltage value of the test voltage TV is smaller than the lower limit Vrmin of the reference range RR. For example, when the test voltage TV has a voltage value that does not reach 0.9V, the source current SI may have intensity weaker than 1 μA. As described above, when the source current SI having immoderately weak intensity is generated, the bio-electrical impedance BZ may not be accurately analyzed. Thus, for the second case, the intensity of the source current SI may be adjusted to increase, and the electronic device 100a may operate in the calibration mode to increase the intensity of the source current SI.

When the voltage value of the test voltage TV is included in the reference range RR, the source current SI may have intensity of 1 μA or about 1 μA. In this case, the electronic device 100a may operate in the measurement mode. In the measurement mode, the electronic device 100a outputs the source current SI to, for example, the body 11, and then obtains information of the bio-electrical impedance BZ using the output source current SI.

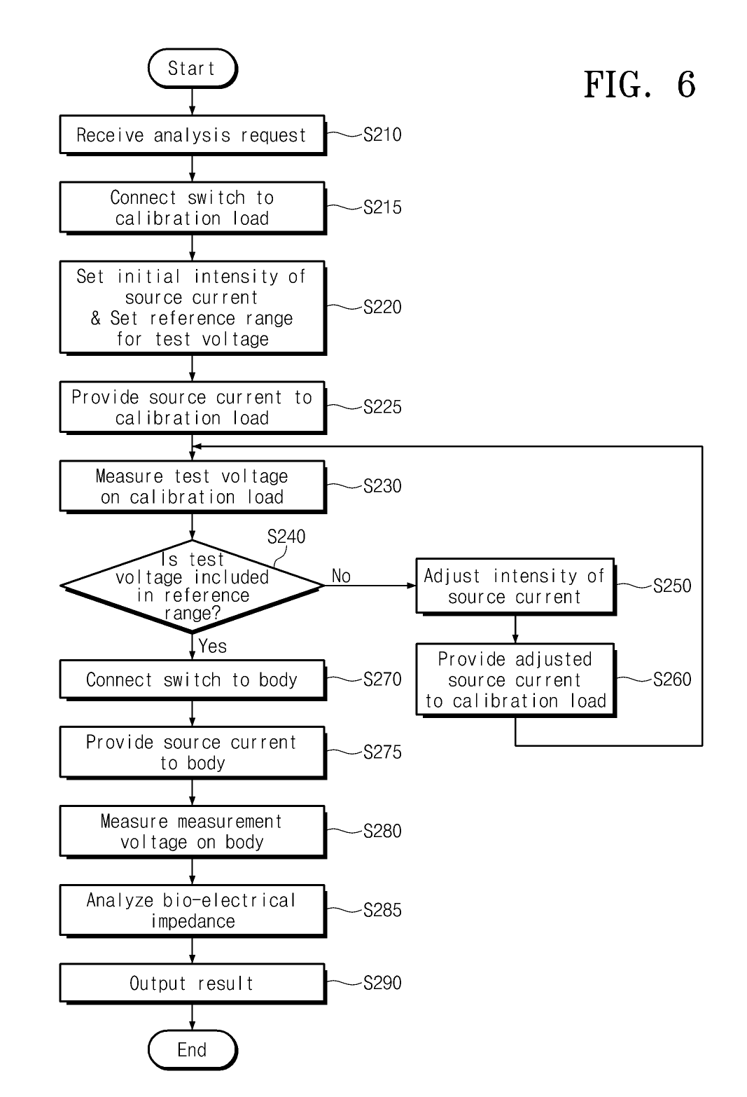

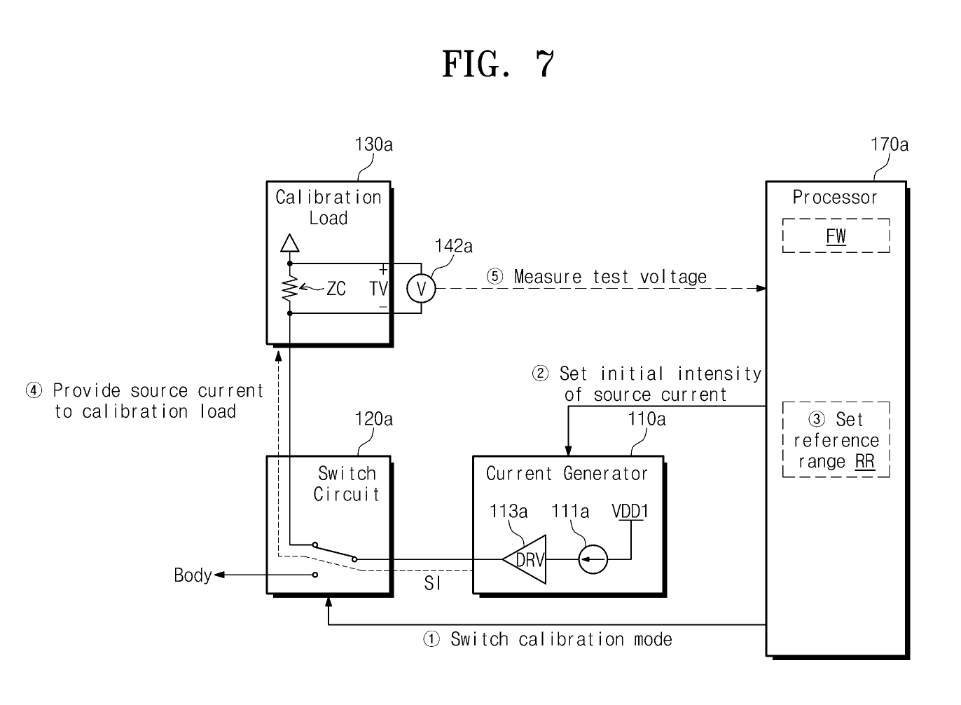

FIG. 6 illustrates a flowchart describing an operation of an electronic device 100a of FIG. 3. FIGS. 7 to 9 illustrate conceptual diagrams describing operations of the electronic device 100a of FIG. 3. To help better understanding, FIGS. 6 through 9 will be referred to together. Further, FIGS. 1 and 3 will be referred to together with FIGS. 6 through 9.

The processor 170a may control operations that will be described below using one or more instruction codes of firmware FW. For example, the processor 170a may load firmware FW stored in the memory 180a.

Referring to FIG. 6, in operation S210, the electronic device 100a receives a request for analyzing the bio-electrical impedance BZ. For example, a user of the electronic device 100a may input the request for analyzing to the electronic device 100a through a user interface of the electronic device 100a. Alternatively or additionally, when a specific condition is satisfied, the request for analyzing may occur inside the electronic device 100a and the processor 170a may recognize the request for analyzing.

Referring to FIGS. 6 and 7, in operation S215, the electronic device 100a operates in the calibration mode, in response to the request for analyzing (refer to operation \textcircled{1} of FIG. 7). According to a control of the processor 170a, the switch circuit 120a connects the current generator 110a to the calibration load 130a. Accordingly, the source current SI is provided to the calibration load 130a.

In operation S220, the processor 170a sets initial intensity of the source current SI such that the current generator 110a will output the source current SI (refer to operation \textcircled{2} of FIG. 7). For example, the processor 170a may set a gain value of the current driver 113a to a default value. Further, the processor 170a may set the reference range RR described with reference to FIG. 5 (refer to operation \textcircled{3} of FIG. 7). For example, the processor 170a may calculate the reference range RR with reference to the reference information RI stored in the memory 180a.

In operation S225, the electronic device 100a provides the source current SI to the calibration load 130a through the switch circuit 120a (refer to operation \textcircled{4} of FIG. 7). For example, processor 170a may control current generator 110a to provide source current SI having the initial intensity set in operation S220. According to the source current SI flowing, the test voltage TV is provided between both ends of the calibration load 130a. Afterwards, in operation S230, the electronic device 100a measures amplitude of the test voltage TV using the voltage meter circuit 142a (refer to operation \textcircled{5} of FIG. 7).

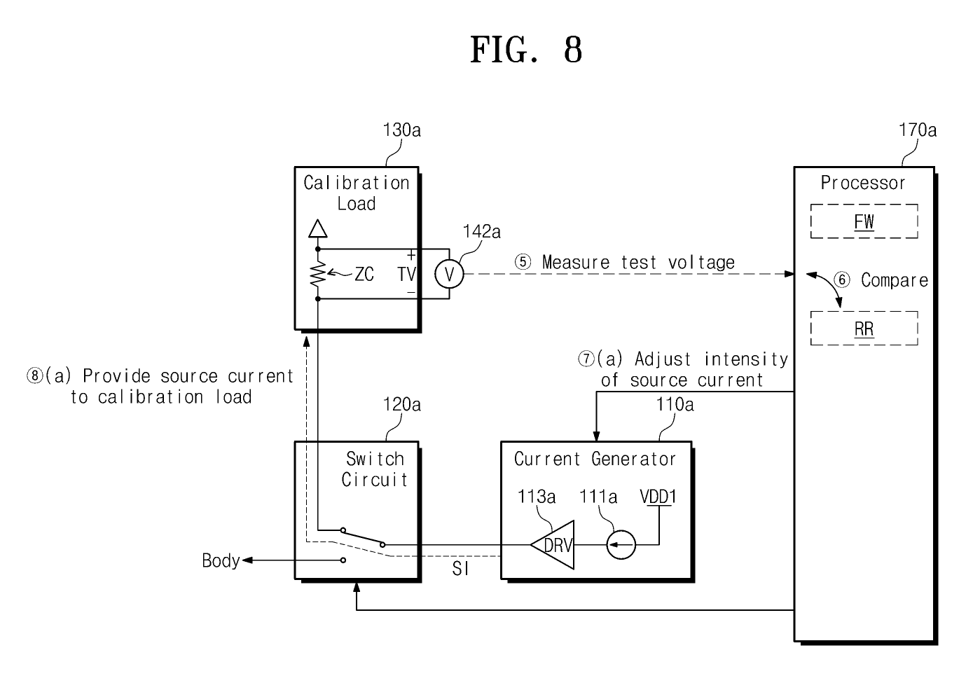

Referring to FIGS. 6 and 8, in operation S240, the processor 170a compares the voltage value of the measured test voltage TV with the reference range RR (refer to operation \textcircled{6} of FIG. 8). For example, the processor 170a may determine whether the voltage value of the test voltage TV is included in the reference range RR. The processor 170a may determine whether the voltage value of the test voltage TV exceeds the upper limit Vrmax or does not reach the lower limit Vrmin. The processor 170a may compare the voltage value of the test voltage TV with each of one or more reference values included in the reference range RR.

The processor 170a may determine in operation S240 that the voltage value of the test voltage TV is not be included in the reference range RR. As described with reference to FIG. 5, the test voltage TV being outside the reference range RR may mean that the intensity of the source current SI is not proper. In this case, in operation S250 the processor 170a subsequently controls the current generator 110a such that the intensity of the source current SI is adjusted (refer to operation \textcircled{7}(a) of FIG. 8). For example, the processor 170a may adjust a gain of the current driver 113a.

Afterwards, in operation S260, the source current SI having the adjusted intensity is provided to the calibration load 130a through the switch circuit 120a (refer to operation \textcircled{8}(a) of FIG. 8). According to the adjusted source current SI, the test voltage TV is provided between both ends of the calibration load 130a. Afterwards, in operation S230, the electronic device 100a measures the amplitude of the test voltage TV that is provided according to the adjusted source current SI using the voltage meter circuit 142a (refer to operation \textcircled{5} of FIG. 8).

When the voltage value of the test voltage TV is not included in the reference range RR, operations S230, S240, S250, and S260 may be repeated. The processor 170a adjusts the intensity of the source current SI until the voltage value of the test voltage TV is included in the reference range RR. The intensity of the source current SI may be repeatedly adjusted according to a control of the processor 170a, such that the voltage value of the test voltage TV is included in the reference range RR.

On the other hand, the processor 170a may determine in operation S240 that the voltage value of the test voltage TV is included in the reference range RR. For example, it may be determined that the voltage value of the test voltage TV is included in the reference range RR responsive to the first occurrence of operation S240. Alternatively, as the intensity of the source current SI is adjusted, the voltage value of the test voltage TV may be changed to be included in the reference range RR. As described with reference to FIG. 5, the test voltage TV included in the reference range RR may mean that the intensity of the source current SI is proper.

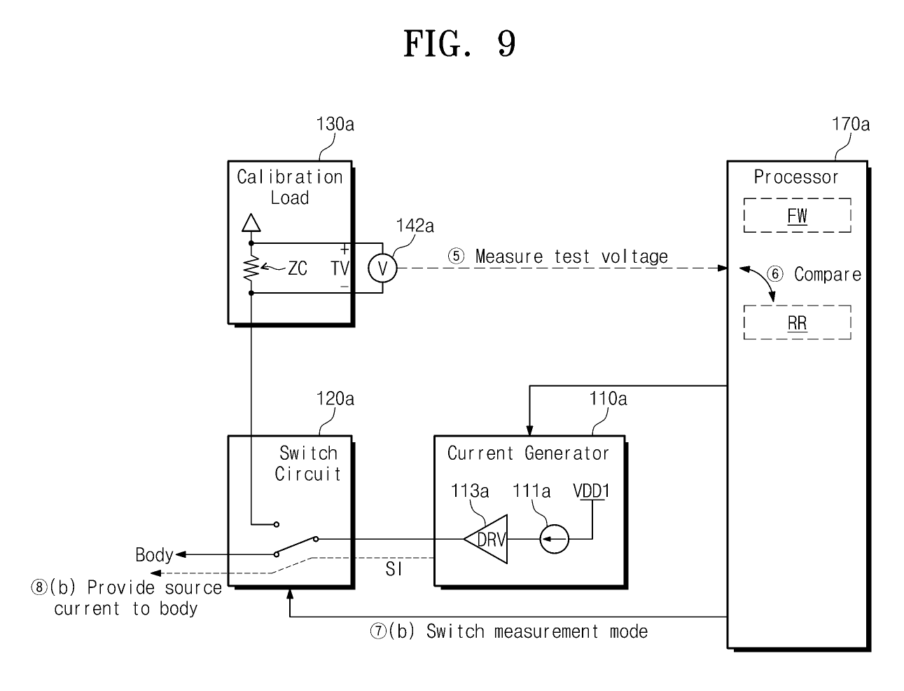

Referring to FIGS. 6 and 9, when the voltage value of the test voltage TV is included in the reference range RR as determined in operation S240, operation S270 is subsequently performed. In operation S270, the electronic device 100a is operated in the measurement mode (refer to operation \textcircled{7} (b) of FIG. 9). According to control of the processor 170a, the switch circuit 120a connects the current generator 110a to the outside of the electronic device 100a (e.g., the body 11). Accordingly, in operation S275, the source current SI is output to the outside of the electronic device 100a (refer to an operation \textcircled{8}(b) of FIG. 9).

In summary, the processor 170a determines whether the intensity of the source current SI is proper, based on the voltage value of the test voltage TV that is provided between both ends of the calibration load 130a according to the source current SI. For example, the processor 170a may determine whether the voltage value of the test voltage TV is included in the reference range RR. As described with reference to FIG. 5, when the voltage value of the test voltage TV is included in the reference range RR, it may be determined that the intensity of the source current SI is proper. On the other hand, when the voltage value of the test voltage TV is not included in the reference range RR, it may be determined that the intensity of the source current SI is not proper.

When it is determined that the intensity of the source current SI is not proper, the processor 170a may operate in the calibration mode. When it is determined that the intensity of the source current SI is proper, the processor 170a may operate in the measurement mode. The processor 170a may control an operation of the switch circuit 120a depending on the operation mode (e.g., at least, the calibration mode or the measurement mode).

Referring to FIG. 6, after the source current SI is output to the outside of the electronic device 100a in operation S270, operation S280 is performed. In operation S280, the electronic device 100a measures the measurement voltage MV using the voltage meter circuit 140. As described with reference to FIG. 1, the measurement voltage MV applied between two electrodes EL3 and EL4, that are connected to the outside of the electronic device 100a (e.g., the body 11), according to the source current SI. A voltage value of the measurement voltage MV may vary depending on the bio-electrical impedance BZ of the body 11.

In operation S285, the processor 170a analyzes the bio-electrical impedance BZ with reference to the measurement voltage MV. The processor 170a may analyze the bio-electrical impedance BZ to obtain information about the bio-electrical impedance BZ. For example, the processor 170a may calculate an impedance value of the bio-electrical impedance BZ. The processor 170a may obtain additional information, such as body fat, muscle, and/or the like, of the body 11, based on the impedance value of the bio-electrical impedance BZ. To achieve this, the memory 180a of the electronic device 100a may store information associated with a correspondence relationship between height, weight, and/or an impedance value of the bio-electrical impedance of the body 11 and body fat and/or muscle of the body 11 in advance before operating the electronic device 100a.

For example, the processor 170a may generate analysis data based on the obtained information. The analysis data may include information and additional information of the bio-electrical impedance BZ. In operation S290, the electronic device 100a outputs the analysis data.

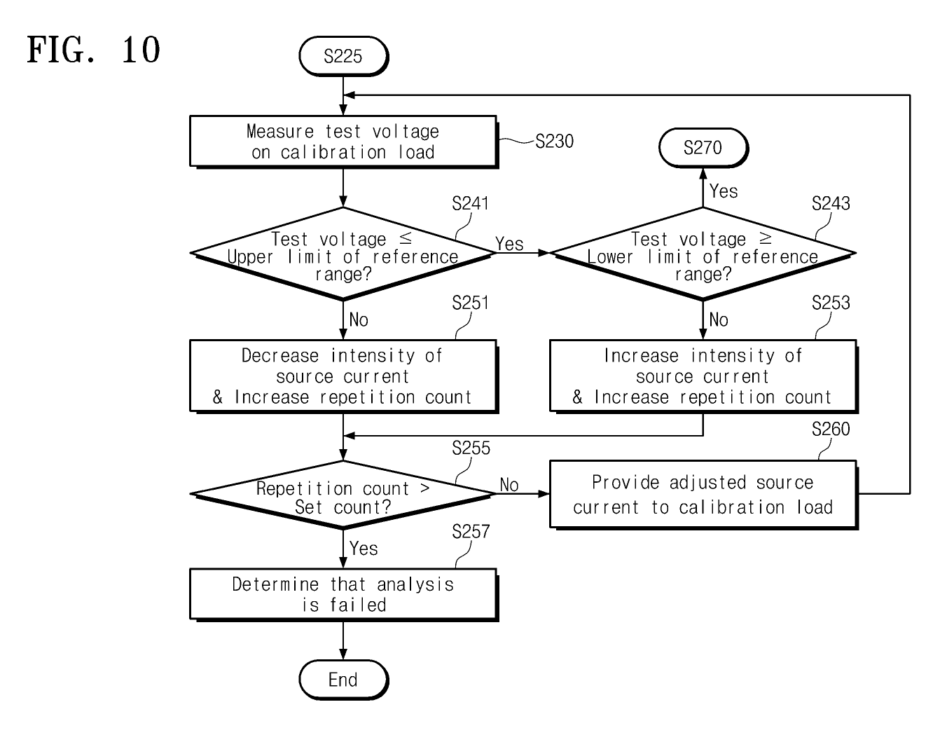

FIG. 10 is a flowchart describing an operation of an electronic device 1010a of FIG. 3. The flowchart of FIG. 10 describes operations S230, S240, S250 and S260 of FIG. 6 in further detail. To help better understanding, FIGS. 1 and 3 will be referred to together with FIG. 10.

Operations of FIG. 10 are performed after operation S225 of FIG. 6. In operation S230 after operation S225, as described with reference to FIG. 6, the electronic device 100a may measure the test voltage TV using the voltage meter circuit 142a.

In operation S241, the processor 170a determines whether a voltage value of the test voltage TV is equal to or smaller than the upper limit Vrmax of the reference range RR. When the voltage value of the test voltage TV is equal to or smaller than the upper limit Vrmax of the reference range RR, operation S243 is performed. In operation S243, the processor 170a determines whether the voltage value of the test voltage TV is equal to or larger than the lower limit Vrmin of the reference range RR.

When the voltage value of the test voltage TV is equal to or smaller than the upper limit Vrmax and is equal to or greater than the lower limit Vrmin, the voltage value of the test voltage TV is included in the reference range RR. In this case, operation S270 as previously described with respect to FIG. 6 is performed, whereby the electronic device 100a operates in the measurement mode.

The processor 170a may determine in operation S241 that the voltage value of the test voltage TV exceeds the upper limit Vrmax of the reference range RR. In this case, operation S251 is performed. In operation S251, the processor 170a controls the current generator 110a such that the intensity of the source current SI decreases.

As described with reference to FIG. 5, when the voltage value of the test voltage TV is greater than the upper limit Vrmax of the reference range RR, the intensity of the source current SI may be excessively strong. The source current SI having strong intensity may damage the body 11. Thus, the intensity of the source current SI may be adjusted to decrease.

In operation S251, a value of “repetition count” is increased and the intensity of the source current SI is decreased. Herein, the “repetition count” may mean the number of times that a process of adjusting the intensity of the source current SI is repeated. For example, the value of repetition count may increase by 1 whenever the processor 170a performs a process of decreasing the intensity of the source current SI. Information of the repetition count may be stored in the memory 180a and/or an internal memory (e.g., a cache) of the processor 170a.

The processor 170a may determine in operation S243 that the voltage value of the test voltage TV does not reach the lower limit Vrmin of the reference range RR. In this case, operation S253 is performed. In operation S253, the processor 170a control the current generator 110a such that the intensity of the source current SI increases.

As described with reference to FIG. 5, when the voltage value of the test voltage TV is smaller than the lower limit Vrmin of the reference range RR, the intensity of the source current SI may be immoderately weak. When the source current SI having weak intensity is generated, the bio-electrical impedance BZ may not be accurately analyzed. Thus, the intensity of the source current SI may be adjusted to increase.

In operation S253, the value of repetition count increases and the intensity of the source current SI is increased. For example, the value of repetition count may increase by 1 whenever the processor 170a performs a process of increasing the intensity of the source current SI.

In operation S255, the processor 170a determines whether the value of the repetition count that is increased in the operation S251 or S253 is larger than a set count. Operation S255 may correspond to operation S170 described with reference to FIG. 4. The value of the repetition count being larger than the set count may mean that adjustment of the intensity of the source current SI has been repeated too many times.

For example, when a calibration operation is continuously performed even though calibrating the source current SI is difficult due to an error of the electronic device 100a, a deadlock may occur in an operation of the electronic device 100a. To avoid the deadlock, the processor 170a may be limited to performing a process of adjusting the intensity of the source current SI as many times as the set count.

For example, the set count may have an appropriate value to avoid the deadlock. For example, the value of the set count may be stored in the memory 180a, and may be referred by the processor 170a. Alternatively or additionally, the value of the set count may be inserted into the instruction code of firmware FW, and may be processed by the processor 170a.

A repetition count that is determined in operation S255 to be larger than the set count may mean that the process of adjusting the intensity of the source current SI has been performed a number of times more than the set count (i.e., adjustment of the intensity of the source current SI has been repeated too many times). When the repetition count is larger than the set count, operation S257 is performed. In operation S257, the processor 170a determines that analyzing the bio-electrical impedance BZ has failed. The electronic device 100a may provide a user with any corresponding result indicating that the analysis has failed.

On the other hand, when a repetition count is determined in operation S255 to not be larger than the set count (i.e., adjustment of the intensity of the source current SI has not been sufficiently repeated), operation S260 is performed. In operation S260, the source current SI having the adjusted intensity is provided to the calibration load 130a through the switch circuit 120a. According to the adjusted source current SI, a test voltage TV is provided between both ends of the calibration load 130a. Afterwards, in operation S230, the electronic device 100a measures the amplitude of the test voltage TV using the voltage meter circuit 142a.

In summary, adjusting the intensity of the source current SI may be repeated a number of times that is less than the set count. When the voltage value of the test voltage TV is not included in the reference range RR while the process of adjusting the intensity of the source current SI is repeated, it may be determined that analyzing the bio-electrical impedance BZ has failed. When the process of adjusting the intensity of the source current SI has been repeated a number of times that is more than the set count, it may be determined that analyzing the bio-electrical impedance BZ has failed. However, when the voltage value of the test voltage TV is changed to be included in the reference range RR in response to adjusting the intensity of the source current SI, analyzing the bio-electrical impedance BZ may be performed.

In embodiments of the inventive concept, the electronic device 100a may include the calibration load 130a having an electrical characteristic that is identical or similar to that of the body 11. Before the source current SI is output to the outside of the electronic device 100a, the source current SI may be provided to the calibration load 130a first. The electronic device 100a may determine whether the intensity of the source current SI is proper, based on the test voltage TV. When the intensity of the source current SI is not proper, the electronic device 100a may calibrate the intensity of the source current SI. When the intensity of the source current SI is proper, the electronic device 100a may output the source current SI to the outside of the electronic device 100a.

According to embodiments, the source current SI used to analyze the bio-electrical impedance BZ may have safe intensity that does not damage the body 11. The source current SI may be calibrated to have intensity that is proper to analyze the bio-electrical impedance BZ or that is requested by a user.

Further, according to embodiments, the electronic device 100a may calibrate the intensity of the source current SI by itself, without separate software or a separate device. Thus, time taken to calibrate the intensity of the source current SI may become shorter, and processing burden due to using the separate software or the separate device may be relieved.

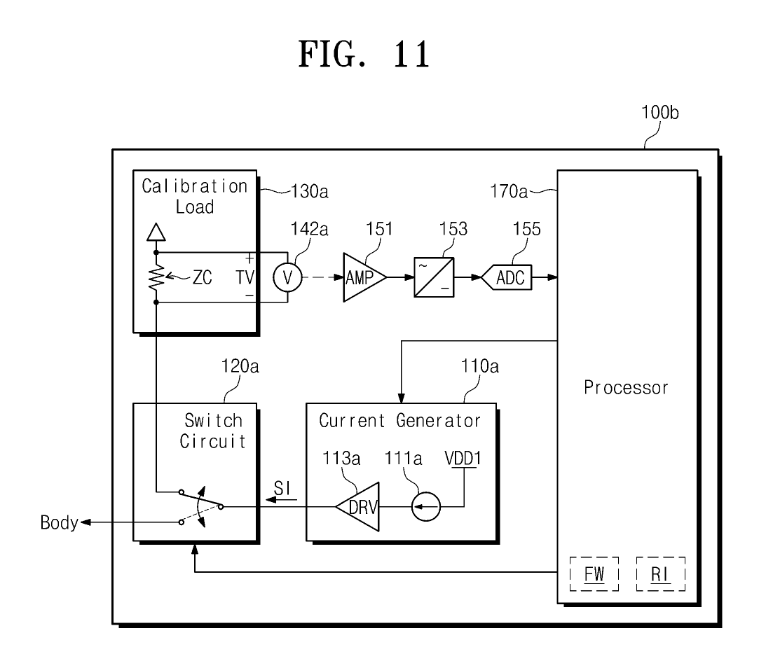

FIG. 11 illustrates a block diagram of an electronic device according to embodiments of the inventive concept. The electronic device 100 of FIG. 1 may include an electronic device 100b of FIG. 11. The electronic device 100b may be used to analyze the bio-electrical impedance BZ of FIG. 1. To help better understanding, FIG. 1 will be referred to together with FIG. 11.

In some embodiments, the electronic device 100b includes a current generator 110a, a switch circuit 120a, a calibration load 130a, a voltage meter circuit 142a, an amplifier 151, an alternating current-to-direct current (AC/DC) converter 153, an analog-to-digital converter (ADC) 155, and a processor 170a. In some embodiments, the electronic device 100b may not include one or more components of FIG. 11, and/or may further include other components that are not illustrated in FIG. 11.

Each of the current generator 110a, the switch circuit 120a, the calibration load 130a, the voltage meter circuit 142a, and the processor 170a may be configured and may operate identically or similarly to those described with reference to FIG. 3. For brevity, redundant descriptions will be omitted below.

In some embodiments, the electronic device 100b may not include the memory 180b of FIG. 3. In such embodiments, data such as an instruction code of firmware FW and reference information RI may be stored in an internal memory (e.g., embedded memory, ROM, and/or the like) of the processor 170a. In some embodiments, the electronic device 100b may include the memory 180a, and the data such as the instruction code of firmware FW and the reference information RI may be dispersively stored in the internal memory of the processor 170a and the memory 180a.

As described above, the source current SI having strong intensity may damage the body 11. Thus, the source current SI may be output to have intensity that is not excessively strong. In this case, the voltage value of the test voltage TV may not be sufficiently large. The amplifier 151 amplifies the amplitude of the test voltage TV such that the voltage value of the test voltage TV is clearly measured. An output of the amplifier 151 is provided to the AC/DC converter 153.

In some embodiments, the source current SI may include an alternating current component. Compared to a direct current component, the alternating current component may have strong energy and thus may be well transmitted to the body 11. In this case, the test voltage TV may include an alternating voltage component. However, since the alternating voltage component has a value that varies according to the lapse of time, it may not be easy to compare the alternating voltage component with the reference range RR. The AC/DC converter 153 converts the alternating voltage component into a direct voltage component such that the comparison operation is easily performed. An output of the AC/DC converter 153 is provided to the ADC 155.

The ADC 155 digitizes the output of the AC/DC converter 153, and outputs a digital value corresponding to the voltage value of the test voltage TV. The processor 170a compares the digital value output from the ADC 155 with the reference range RR.

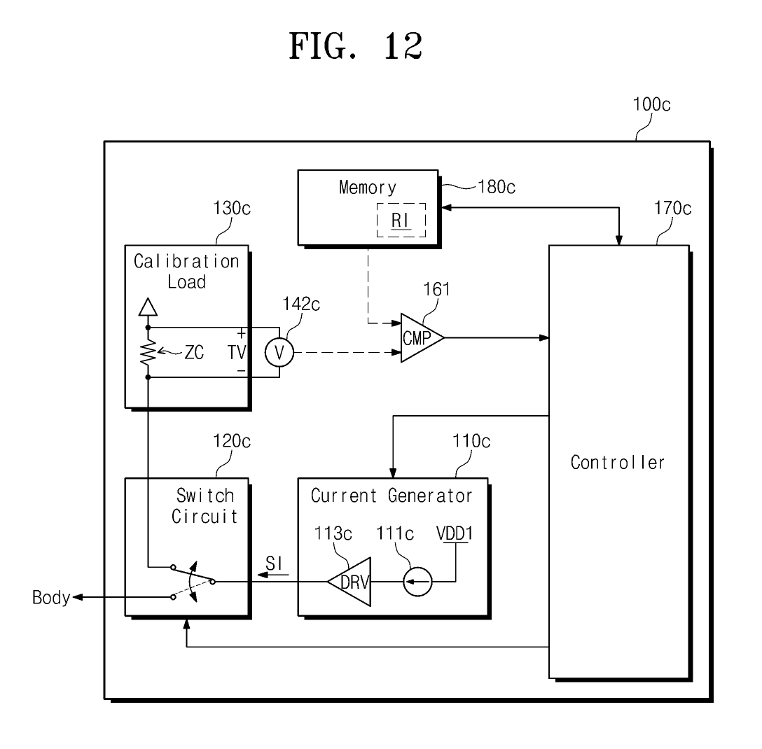

FIG. 12 illustrates a block diagram of an electronic device according to embodiments of the inventive concept. For example, the electronic device 100 of FIG. 1 may include an electronic device 100c of FIG. 12. The electronic device 100c may be used to analyze the bio-electrical impedance BZ of FIG. 1. To help better understanding, FIG. 1 will be referred to together with FIG. 12.

In some embodiments of the inventive concept, the electronic device 100c includes a current generator 110c, a switch circuit 120c, a calibration load 130c, a voltage meter circuit 142c, a comparator 161, a controller 170c, and a memory 180c. In some embodiments, the electronic device 100c may not include one or more components of FIG. 12, and/or may further include other components that are not illustrated in FIG. 12.

The current generator 110c including a current source 111c and a current driver 113c, the switch circuit 120c, the calibration load 130c, the voltage meter circuit 142c, and the memory 180c may be configured and may operate identically or similarly to the current generator 110a, the current source 111a, the current driver 113a, the switch circuit 120a, the calibration load 130a, the voltage meter circuit 142a, and the memory 180a of FIG. 3 respectively. For brevity, redundant descriptions will be omitted below.

The comparator 161 receives information associated with a voltage value of the test voltage TV from the voltage meter circuit 142c. The comparator 161 receives one or more reference values included in the reference range RR from the memory 180c, based on the reference information RI stored in the memory 180c. The reference value may be one of values included in the reference range RR. For example, the reference value may increase by a specific increment from the lower limit Vrmin of the reference range RR to the upper limit Vrmax of the reference range RR. Alternatively, the reference value may decrease by a specific decrement from the upper limit Vrmax of the reference range RR to the lower limit Vrmin of the reference range RR.

The comparator 161 compares each of the reference values with the voltage value of the test voltage TV, and outputs a comparison result. The comparison result may indicate whether the voltage value of the test voltage TV is the same as the reference value. Alternatively, a comparison result may indicate whether the voltage value of the test voltage TV is greater or smaller than the reference value. For example, the comparator 161 may be implemented in a hardware circuit including a plurality of semiconductor elements.

The controller 170c controls the overall operations of the electronic device 100c. For example, the controller 170c may process various arithmetic operations and/or logical operations that are required to operate the electronic device 100c. The controller 170c may include at least one processor core that is capable of processing various operations. The controller 170c may perform some functions of the processor 170a of FIG. 3.

The controller 170c may control the current generator 110c based on an output of the comparator 161. For example, the controller 170c may control the current generator 110c to control the intensity of the source current SI. In the calibration mode, the controller 170c adjusts the intensity of the source current SI such that the source current SI has proper intensity, based on the output of the comparator 161. The controller 170c provides a control signal(s) to the current generator 110c to adjust the intensity of the source current SI.

The controller 170c may control an operation of the switch circuit 120c based on the output of the comparator 161. In the calibration mode, the controller 170c controls the switch circuit 120c such that the source current SI is provided to the calibration load 130c. In the measurement mode, the controller 170c controls the switch circuit 120c such that the source current SI is output to the outside of the electronic device 100c (e.g., the body 11).

In the embodiment of FIG. 3, most operations for managing and controlling the electronic device 100a may be processed by the processor 170a. In the embodiment of FIG. 12, the controller 170c may process the minimum scope of operations for managing and controlling the electronic device 100c. Instead, the electronic device 100c may include other components configured to perform some functions of the processor 170a. For example, the comparison operation of the processor 170a in the embodiment of FIG. 3 may be performed by the comparator 161 in the embodiment of FIG. 12, instead of by the controller 170c.

For example, when the output of the comparator 161 indicates that the voltage value of the test voltage TV is greater than the upper limit Vrmax or smaller than the lower limit Vrmin, the controller 170c may determine that the reference range RR does not include the voltage value of the test voltage TV. This, as described with reference to FIG. 5, may mean that the intensity of the source current SI is not proper. Thus, for the calibration mode, the source current SI may be provided to the calibration load 130c through the switch circuit 120c.

In the calibration mode, the intensity of the source current SI may be adjusted. Adjusting the intensity of the source current SI may be repeated until the output of the comparator 161 indicates that the reference range RR includes the voltage value of the test voltage TV.

Meanwhile, when the output of the comparator 161 indicates that the voltage value of the test voltage TV is equal to or smaller than the upper limit Vrmax and is equal to or greater than the lower limit Vrmin, the controller 170c may determine that the reference range RR includes the voltage value of the test voltage TV. This, as described with reference to FIG. 5, may mean that the intensity of the source current SI is proper. Thus, for the measurement mode, the source current SI may be provided to the outside of the electronic device 100c through the switch circuit 120c.

After the source current SI is output to the outside of the electronic device 100c, the electronic device 100c may measure the measurement voltage MV by using the voltage meter circuit 140. The controller 170c may obtain information of the bio-electrical impedance BZ with reference to the measurement voltage MV. For example, the controller 170c may calculate an impedance value of the bio-electrical impedance BZ. The controller 170c may obtain additional information, such as body fat, muscle, and/or the like, of the body 11, based on the impedance value of the bio-electrical impedance BZ. The controller 170c may generate analysis data based on the obtained information. The electronic device 100c may provide the analysis data to a user.

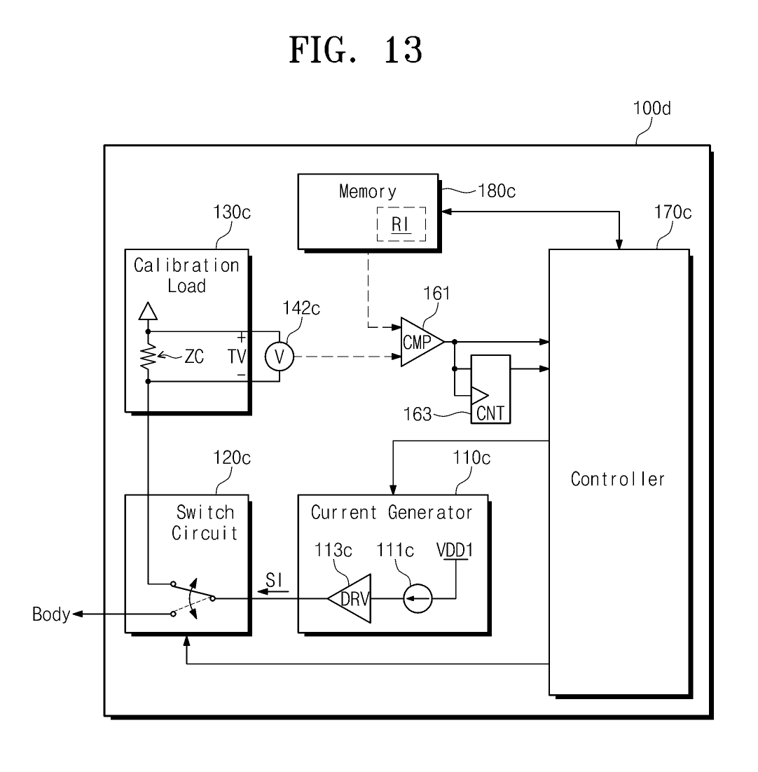

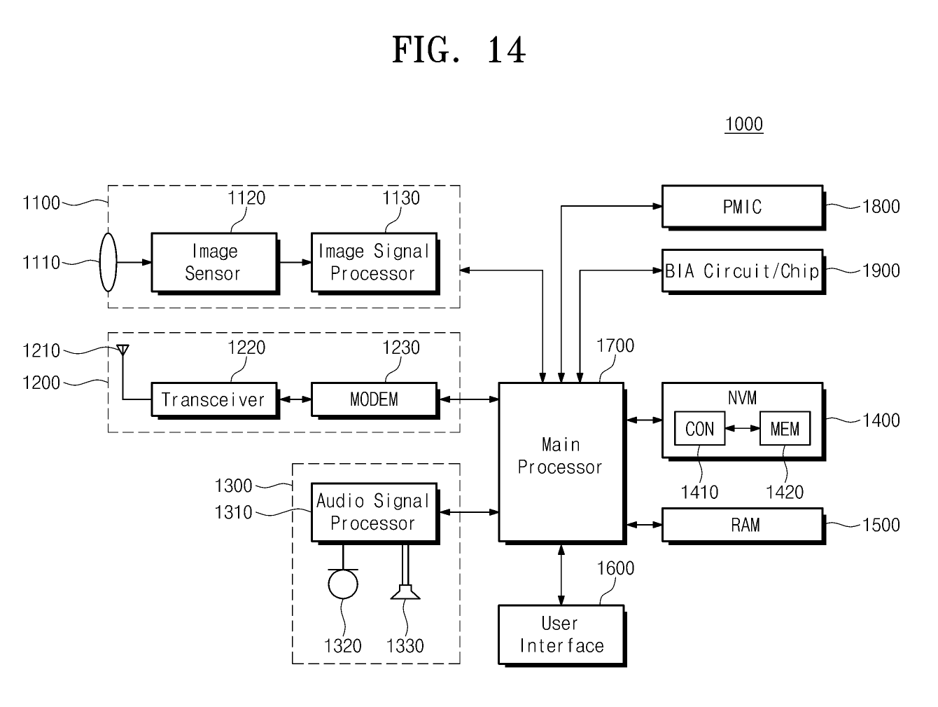

FIG. 13 illustrates a block diagram of an electronic device according to embodiments of the inventive concept. The electronic device 100 of FIG. 1 may include an electronic device 100d of FIG. 13. The electronic device 100d may be used to analyze the bio-electrical impedance BZ of FIG. 1. To help better understanding, FIG. 1 will be referred to together with FIG. 13.

In some embodiments, the electronic device 100d includes a current generator 110c, a switch circuit 120c, a calibration load 130c, a voltage meter circuit 142c, a comparator 161, a counter 163, a controller 170c, and a memory 180c. In some embodiments, the electronic device 100d may not include one or more components of FIG. 13, and/or may further include other components that are not illustrated in FIG. 13.

The current generator 110c, a current source 111c, a current driver 113c, the switch circuit 120c, the calibration load 130c, the voltage meter circuit 142c, and the memory 180c may be configured and may operate identically or similarly to the current generator 110a, the current source 111a, the current driver 113a, the switch circuit 120a, the calibration load 130a, the voltage meter circuit 142a, and the memory 180a of FIG. 3 respectively. Each of the comparator 161 and the controller 170c may be configured and may operate identically or similarly to those described with reference to FIG. 12. For brevity, redundant descriptions will be omitted below.

The counter 163 counts a repetition count whereby a process of adjusting the intensity of the source current SI is repeated. As described with reference to operation S170 of FIG. 4 and operation S255 of FIG. 10, in some embodiments, the controller 170c of the electronic device 100d manages the number of times adjustment of the intensity of the source current SI is repeated based on the repetition count provided by counter 163. For example, a value of the repetition count stored in the counter 163 may increase by 1 whenever the process of adjusting the intensity of the source current SI is performed.

Adjusting the intensity of the source current SI is performed by the controller 170c while the repetition count is equal to or smaller than a set count. Adjusting the intensity of the source current SI is repeated as many times as the set count. When the repetition count exceeds the set count, the controller 170c determines that analyzing the bio-electrical impedance BZ has failed. Thus, an operation deadlock of the electronic device 100d may be prevented.

FIG. 14 illustrates a block diagram of a mobile electronic device that includes a bio-electrical impedance analysis circuit/chip according to embodiments of the inventive concept. A mobile electronic device 1000 includes an image processor 1100, a wireless communication block 1200, an audio processor 1300, a nonvolatile memory 1400, a RAM 1500, a user interface 1600, a main processor 1700, a power management integrated circuit 1800, and a bio-electrical impedance analysis (BIA) circuit/chip 1900. For example, the mobile electronic device 1000 may be one of a mobile terminal, a portable digital assistant (PDA), a personal multimedia player (PMP), a digital camera, a smart phone, a tablet computer, a wearable device, and/or the like.

The image processor 1100 may receive light through a lens 1110. An image sensor 1120 and an image signal processor 1130 included in the image processor 1100 generate an image based on the received light.

The wireless communication block 1200 includes an antenna 1210, a transceiver 1220, and a modulator/demodulator (MODEM) 1230. The wireless communication block 1200 may communicate with the outside of the mobile electronic device 1000 in compliance with various wireless communication protocols, such as global system for mobile communication (GSM), code division multiple access (CDMA), wideband CDMA (WCDMA), high speed packet access (HSPA), evolution-data optimized (EV-DO), worldwide interoperability for microwave access (WiMax), wireless broadband (WiBro), long term evolution (LTE), Bluetooth, near field communication (NFC), wireless fidelity (WiFi), radio frequency identification (RFID), and/or the like.

The audio processor 1300 processes an audio signal using the audio signal processor 1310. The audio processor 1300 may receive an audio input through a microphone 1320, and/or provide an audio output through a speaker 1330.

The nonvolatile memory 1400 may store data that is required to be retained regardless of power supply. For example, the nonvolatile memory 1400 may include at least one of flash memory, PRAM, MRAM, ReRAM, FRAM, and/or the like. According to a control of a memory controller 1410, a memory device 1420 may store data and/or may output data.

The RAM 1500 may store data used to operate the mobile electronic device 1000. For example, the RAM 1500 may operate as a working memory, an operation memory, and/or a buffer memory of the mobile electronic device 1000. The RAM 1500 may temporarily store data processed or to be processed by the main processor 1700.

The user interface 1600 may process interfacing between a user and the mobile electronic device 1000 according to a control of the main processor 1700. The user interface 1600 may include an input interface, such as a keyboard, a keypad, a button, a touch panel, a touch screen, a touch pad, a touch ball, a camera, a microphone, a gyroscope sensor, a vibration sensor, and/or the like. The user interface 1600 may include an output interface, such as a display device, a motor, and/or the like. The display device may include at least one of a liquid crystal display (LCD), a light emitting diode (LED) display, an organic LED (OLED) display, an active matrix OLED (AMOLED) display, and/or the like.

The main processor 1700 may control the overall operations of the mobile electronic device 1000. The image processor 1100, the wireless communication block 1200, the audio processor 1300, the nonvolatile memory 1400, and the RAM 1500 may perform a user command provided through the user interface 1600 according to a control of the main processor 1700 and/or may provide a service to a user through the user interface 1600 according to a control of the main processor 1700. The main processor 1700 may be implemented in a system on chip (SoC). For example, the main processor 1700 may include an application processor.

The power management integrated circuit 1800 may manage power used to operate the mobile electronic device 1000. The power management integrated circuit 1800 may appropriately convert power provided from a battery (not shown) or an external power supply (not shown). Further, the power management integrated circuit 1800 may provide the converted power to components of the mobile electronic device 1000.

The BIA circuit/chip 1900 may be used to analyze bio-electrical impedance. The BIA circuit/chip 1900 may be configured and may operate based on the example embodiments described with reference to FIGS. 1 through 13.

For example, the BIA circuit/chip 1900 may include a calibration load having an electrical characteristic that is identical or similar to that of a body. The BIA circuit/chip 1900 may operate in a calibration mode in response to a request of analyzing the bio-electrical impedance. During the calibration mode, intensity of source current may be calibrated. When the intensity of the source current is proper, the BIA circuit/chip 1900 may obtain information of the bio-electrical impedance of the body by using the source current, in a measurement mode. For brevity, redundant descriptions associated with the example embodiments will be omitted below.

According to embodiments of the inventive concept, the source current used to analyze the bio-electrical impedance may have safe intensity. The source current may also be calibrated to have intensity that is proper to analyze the bio-electrical impedance or intensity requested by a user. Further, the BIA circuit/chip 1900 may calibrate the intensity of the source current SI by itself, without separate software or a separate device. Thus, time being taken to calibrate the intensity of the source current SI may become shorter, and processing burden due to using the separate software or the separate device may be relieved.

The circuit, the chip, and/or the device in accordance with the embodiments may be mounted using various types of packages, such as package on package (PoP), ball grid arrays (BGAs), chip scale packages (CSPs), plastic leaded chip carrier (PLCC), plastic dual in-line package (PDIP), die in waffle pack, die in wafer form, chip on board (COB), ceramic dual in-line package (CERDIP), metric quad flat pack (MQFP), thin quad flat pack (TQFP), small outline integrated circuit (SOIC), shrink small outline package (SSOP), thin small outline package (TSOP), system in package (SIP), multi-chip package (MCP), wafer-level fabricated package (WFP), wafer-level processed stack package (WSP), and/or the like.

The inventive concepts have been described based on the above example embodiments. However, the inventive concepts may be achieved in different manners, and it should be understood that the described embodiments are illustrative views and not limiting. Accordingly, modified or altered embodiments that do not depart from the spirit or scope of the inventive concepts should be included in the scope of the claims below. That is, the scope of the present disclosure is not limited to the above example embodiments.

What is claimed is:

1. An electronic device configured to analyze bio-electrical impedance, the electronic device comprising:
a current generator configured to generate source current;
a calibration load comprising an impedance component;
a switch circuit configured to selectively provide the source current to the calibration load and to output the source current externally of the electronic device; and
a processor configured to control the switch circuit to provide the source current to the calibration load in response to a request for analyzing the bio-electrical impedance, and to output the source current externally of the electronic device upon determination that a voltage value of a test voltage is within a reference range, the test voltage provided between both ends of the calibration load responsive to the source current.

2. The electronic device of claim 1, wherein an impedance value of the impedance component corresponds to an estimated impedance value of the bio-electrical impedance.

3. The electronic device of claim 1, wherein the switch circuit is configured to be connected to a body including the bio-electrical impedance when the source current is output externally of the electronic device.

4. The electronic device of claim 1, wherein the processor is further configured to control the current generator to adjust intensity of the source current when the voltage value of the test voltage is not within the reference range.

5. The electronic device of claim 4, wherein the switch circuit is configured to provide the source current having the adjusted intensity to the calibration load, and
wherein the processor is further configured to control an operation of the switch circuit based on whether a voltage value of the test voltage that is provided between the both ends of the calibration load according to the source current having the adjusted intensity is within the reference range.

6. The electronic device of claim 4, wherein the processor is further configured to control the current generator to decrease the intensity of the source current upon determination that the voltage value of the test voltage exceeds an upper limit of the reference range, and to increase the intensity of the source current upon determination that the voltage value of the test voltage is below a lower limit of the reference range.

7. The electronic device of claim 4, wherein the processor is further configured to control the current generator to repeatedly adjust the intensity of the source current until the voltage value of the test voltage is within the reference range.

8. The electronic device of claim 7, wherein the processor is further configured to control the current generator to repeatedly adjust the intensity of the source current a number of times equal to or less than a set count, and
wherein the processor is further configured to determine that analyzing the bio-electrical impedance has failed upon determination that the voltage value of the test voltage that is provided between the both ends of the calibration load according to the source current having the adjusted intensity is not within the reference range after repeatedly adjusting the intensity of the source current as the number of times equal to the set count.

9. The electronic device of claim 1, wherein after the source current is output externally of the electronic device, the processor is further configured to obtain information associated with the bio-electrical impedance with reference to a voltage applied between two electrodes responsive to the output source current, the two electrodes being connected to an outside of the electronic device.

10. An electronic device configured to analyze bio-electrical impedance, the electronic device comprising:
a calibration load comprising an impedance component;
a switch circuit configured to selectively provide a source current to the calibration load and to output the source current externally of the electronic device, the source current being generated by a current generator;
a comparator configured to compare a voltage value of a test voltage with one or more reference values, the test voltage provided between both ends of the calibration load responsive to the source current provided from the switch circuit, the one or more reference values being included in a reference range; and
a controller configured to control an operation of the switch circuit and an intensity of the source current generated by the current generator, based on an output of the comparator.

11. The electronic device of claim 10, wherein the switch circuit is configured to provide the source current to the calibration load when the output of the comparator indicates that the voltage value of the test voltage is not within the reference range, according to control of the controller, and
wherein the switch circuit is configured to output the source current externally of the electronic device when the output of the comparator indicates that the voltage value of the test voltage is within the reference range, according to control of the controller.

12. The electronic device of claim 10, wherein the controller is further configured to provide a control signal to the current generator to adjust the intensity of the source current when the output of the comparator indicates that the voltage value of the test voltage is not within the reference range.

13. The electronic device of claim 12, wherein the controller is configured to repeatedly adjust the intensity of the source current until the output of the comparator indicates that the voltage value of the test voltage is within the reference range or until a number of times the intensity of the source current is repeatedly adjusted is equal to a set count.

14. The electronic device of claim 13, further comprising a counter configured to count the number of times the intensity of the source current is repeatedly adjusted.

15. The electronic device of claim 10, wherein the controller is further configured to obtain information associated with the bio-electrical impedance with reference to a voltage applied between two electrodes responsive to the source current output externally of the electronic device, the two electrodes being connected to an outside of the electronic device, and to generate analysis data based on the obtained information.

16. An electronic device configured to analyze bio-electrical impedance, the electronic device comprising:
a current generator configured to generate a source current;
a calibration load comprising an impedance component and configured to provide a test voltage responsive to the source current, wherein an impedance value of the impedance component corresponds to an estimated impedance value of the bio-electrical impedance;
a pair of electrodes connected to an outside of the electronic device; and
a processor configured to control the current generator to adjust an intensity of the source current responsive to the test voltage, to output the source current having the adjusted intensity externally of the electronic device, and to obtain information associated with the bio-electrical impedance based on a voltage externally applied to the pair of electrodes responsive to the output source current.

17. The electronic device of claim 16, further comprising a switch circuit configured to provide the source current to the calibration load during a calibration mode and to output the source current having the adjusted intensity externally of the electronic device responsive to the processor.

18. The electronic device of claim 16, wherein the impedance value is adjustable responsive to the processor.

19. The electronic device of claim 16, wherein the processor is further configured to control the current generator to repeatedly adjust the intensity of the source current to be within a reference range.

20. The electronic device of claim 19, wherein the processor is further configured to control the current generator to repeatedly adjust the source current a number of times equal to or less than a set count, and to determine that analyzing the bio-electrical impedance has failed upon determination that the source current having the adjusted intensity is not within the reference range after the number of times equals the set count.
